(19) United States
(12) Patent Application Publication
(10) Pub. No.: US 2021/0145658 A1
(43) Pub. Date: May 20, 2021

(54) ABSORBENT BODY FOR USE IN AN ABSORBENT ARTICLE

(71) Applicant: Kimberly-Clark Worldwide, Inc., Neenah, WI (US)

(72) Inventors: SungSu Kim, Suji-gu, Yongin-si (KR); KueYoung You, Sujeong-gu, Seongnam-si (KR); JuHyung Lee, Youngtong-gu, Suwon-si (KR); SeoYeon Son, Bundang-gu, Seongnam-si (KR); EunSoo Cho, Bundang-gu, Seongnam-si (KR)

(21) Appl. No.: 17/048,366

(22) PCT Filed: Apr. 19, 2018
(86) PCT No.: PCT/US2018/028352

§ 371 Date: Oct. 16, 2020

(51) Int. Cl. A61F 13/533

(52) U.S. Cl. CPC A61F 13/533

(57) ABSTRACT

An absorbent body for use in an absorbent article can have an improved vertical wicking capability and an improved liquid distribution capability. The absorbent body can have at least a first absorbent layer and a second absorbent layer. The absorbent body can further have at least three pairs of compressed points. A first pair of compressed points can be separated from a second pair of compressed points by a first distance in the longitudinal direction of the absorbent body. A third pair of compressed points can be separated from the first pair of compressed points by a second distance in the transverse direction of the absorbent article. The second distance can be greater than the first distance.

### BACKGROUND

Products such as absorbent articles are often used to collect and retain human body exudates containing, for example, urine, menses and/or blood. Comfort, absorbency, and discretion are three main product attributes and areas of concern for the wearer of the product. In particular, a wearer is often interested in knowing that such products will absorb significant volumes of body exudates with minimal leakage in order to protect their undergarments, outer garments, or bedsheets from staining, and that such products will help them avoid the subsequent embarrassment brought on by such staining.

Currently, a wide variety of products for absorption of body exudates are available in the form of feminine pads, sanitary napkins, panty shields, pantiliners, and incontinence devices. These products generally have an absorbent core positioned between a body-facing liquid permeable topsheet layer and a garment-facing liquid impermeable backsheet layer. The edges of the topsheet and the backsheet layers are often bonded together at their periphery to form a seal to contain the absorbent core and body exudates received into the product through the topsheet layer. In use, such products are typically positioned in the crotch portion of an undergarment for absorption of the body exudates and a garment attachment adhesive on the backsheet layer can be used to attach the product to the inner crotch portion of the undergarment. Some of these products can also include wing-like structures for wrapping about the wearer's undergarment to further secure the product to the undergarment and to protect the undergarment from staining. Such wing-like structures (also known as flaps or tabs) are frequently made from lateral extensions of the topsheet and/or backsheet layers.

One problem with such conventional absorbent articles is that the body exudates are usually not well absorbed by the absorbent article. The body exudates may remain on and/or within an upper layer of the absorbent article rather than moving towards a lower layer of the absorbent article. Body exudates remaining on and/or within an upper layer of the absorbent article can result in a feeling of wetness and discomfort for the wearer of the absorbent article. This can ultimately lead to a wearer of the absorbent article having a feeling of early failure of the absorbent article as the body exudates to be absorbed cannot be efficiently spread throughout the absorbent article. If the body exudates to be absorbed cannot be efficiently spread through the absorbent article, they may run off the edge of the absorbent article causing leakage and staining.

As a result, there remains a need for an improved absorbent body for use in an absorbent article that has an improved vertical wicking capability and an improved liquid distribution capability.

### SUMMARY

In various embodiments, an absorbent body can have a longitudinal direction and a transverse direction; a first absorbent layer; a second absorbent layer; a first pair of compressed points separated in the longitudinal direction from a second pair of compressed points by a first distance; a third pair of compressed points separated in the transverse direction from the first pair of compressed points by a second distance. In various embodiments, the second distance is the same as the first distance. In various embodiments, the second distance is greater than the first distance. In various embodiments, each compressed point of the first pair of compressed points is separated from each other by a third distance which is smaller than the first distance.

In various embodiments, the absorbent body can further have a third absorbent layer.

In various embodiments, the first pair of compressed points are oriented in the longitudinal direction. In various embodiments, the second pair of compressed points are oriented in the longitudinal direction. In various embodiments, the third pair of compressed points are offset from the first pair of compressed points.

In various embodiments, the first pair of compressed points has a leading edge and a trailing edge separated by a fourth distance. In various embodiments, the fourth distance is the same as the first distance. In various embodiments, the first distance is greater than the fourth distance.

In various embodiments, the proportion of the area of the compressed points with respect to the area of the absorbent body is less than about 12%.

In various embodiments, the first absorbent layer is a through-air bonded carded web, the second absorbent layer is a cellulosic fluff based material, and the third absorbent layer is a tissue layer.

In various embodiments, each compressed point of the first pair of compressed points, the second pair of compressed points, and the third pair of compressed points extends from an uppermost surface of the absorbent body to a bottommost surface of the absorbent body.

In various embodiments, an absorbent article can have a topsheet layer; a backsheet layer; and the absorbent body described herein. In various embodiments, the absorbent article can further have a fluid intake layer positioned between the absorbent body and the topsheet layer.

### BRIEF DESCRIPTION OF THE DRAWINGS

FIG. 1 is a top down view of an embodiment of an absorbent body.

FIG. 2A is a cross-sectional view of an embodiment of the absorbent body of FIG. 1 taken along line 2A-2A.

FIG. 2B is a cross-sectional view of another embodiment of the absorbent body of FIG. 1 taken along line 2B-2B.

FIG. 3 is a perspective view of an embodiment of an absorbent article.

FIG. 4 is an exploded perspective view of an embodiment of an absorbent article.

FIG. 5 is an exploded perspective view of an embodiment of an absorbent article.

FIG. 6 is an exploded perspective view of an embodiment of an absorbent article.

### DETAILED DESCRIPTION

The present disclosure is generally directed towards an absorbent body for use in an absorbent article which can have an improved vertical wicking capability and an improved liquid distribution capability. An absorbent body can have a longitudinal direction and a transverse direction. In various embodiments, the absorbent body can have at least a first absorbent layer and a second absorbent layer. In various embodiments, the absorbent body can have a third absorbent layer. The absorbent body can further have at least three pairs of compressed points. A first pair of compressed points can be separated from a second pair of compressed points by a first distance in the longitudinal direction of the absorbent body. A third pair of compressed points can be separated from the first pair of compressed points by a second distance in the transverse direction of the absorbent article. The second distance can be greater than the first distance. Each of the compressed points can provide a pathway for body exudates to travel from the upper surface of the absorbent body and into the lower layer(s) of the absorbent body. Providing pathways for the body exudates to travel can increase the vertical wicking capability of the absorbent body and decrease the feeling of wetness to the wearer of the absorbent article. In various embodiments, the pattern of the compressed points can more effectively contain the body exudates in a localized area of the absorbent body thereby reducing spread of the body exudates in at least the transverse direction of the absorbent body which can result in a reduction of incidents of leakage of body exudates from the absorbent article.

### Definitions

As used herein, the term “absorbent article” refers herein to a garment or other end-use personal care absorbent article, including, but not limited to, catamenial products, such as sanitary napkins, feminine pads, pantiliners, and panty shields, incontinence devices, and the like.

As used herein, the term “airlaid” refers herein to a web manufactured by an airlaying process. In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers herein to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “coform” refers herein to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff, and also superabsorbent particles, inorganic and/or organic absorbent materials, treated polymeric staple fibers and so forth. Some examples of such coform materials are disclosed in U.S. Pat. No. 4,100,324 to Anderson, et al., U.S. Pat. No. 4,818,464 to Lau, U.S. Pat. No. 5,284,703 to Everhart, et al., and U.S. Pat. No. 5,350,624 to Georger, et al., each of which are incorporated herein in their entirety by reference thereto for all purposes.

As used herein, the term “conjugate fibers” refers herein to fibers which have been formed from at least two polymer sources extruded from separate extruders and spun together to form one fiber. Conjugate fibers are also sometimes referred to as bicomponent fibers or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al. each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10 or 20 gsm to about 120, 125 or 150 gsm.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP's ability to absorb water is based in part on ionicity (a factor of the ionic concentration of the aqueous solution), and the SAP functional polar groups that have an affinity for water. SAP are typically made from the polymerization of acrylic acid blended with sodium hydroxide in the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating on another material or fiber.

### Absorbent Body:

The present disclosure is generally directed towards an absorbent body for use in an absorbent article which can have an improved vertical wicking capability and an improved liquid distribution capability. An absorbent body can have a longitudinal direction and a transverse direction. In various embodiments, the absorbent body can have at least a first absorbent layer and a second absorbent layer. In various embodiments, the absorbent body can have a third absorbent layer. The absorbent body can further have at least three pairs of compressed points. A first pair of compressed points can be separated from a second pair of compressed points by a first distance in the longitudinal direction of the absorbent body. A third pair of compressed points can be separated from the first pair of compressed points by a second distance in the transverse direction of the absorbent article. The second distance can be greater than the first distance. Each of the compressed points can provide a pathway for body exudates to travel from the upper surface of the absorbent body and into the lower layer(s) of the absorbent body. Providing pathways for the body exudates to travel can increase the vertical wicking capability of the absorbent body and decrease the feeling of wetness to the wearer of the absorbent article. In various embodiments, the pattern of the compressed points can more effectively contain the body exudates in a localized area of the absorbent body 10 thereby reducing spread of the body exudates in at least the transverse direction of the absorbent body 10 which can result in a reduction of incidents of leakage of body exudates from the absorbent article.

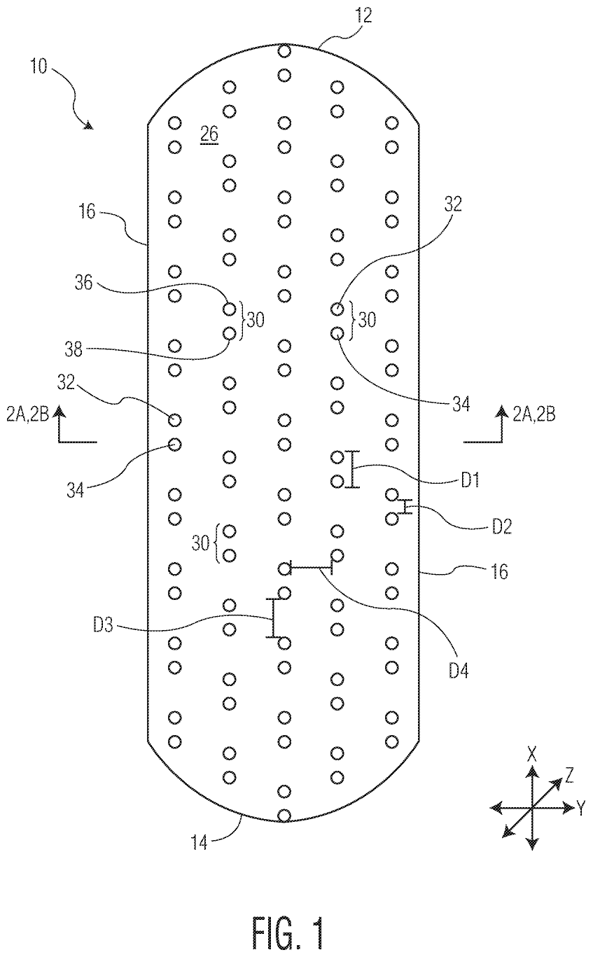

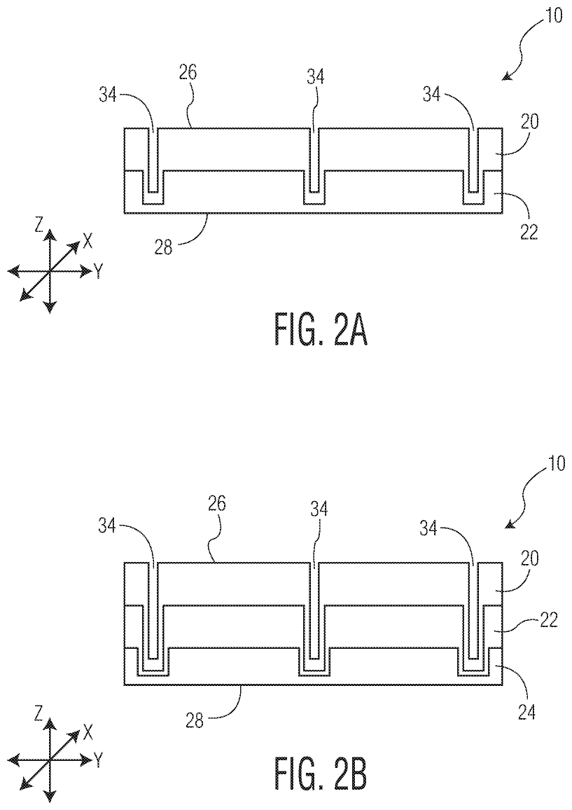

Referring to FIGS. 1, 2A, and 2B, FIG. 1 provides an exemplary illustration of an absorbent body 10, FIG. 2A provides an exemplary illustration of an embodiment of a cross-section of the absorbent body 10 of FIG. 1 taken along line 2A-2A, and FIG. 2B provides an exemplary illustration of another embodiment of a cross-section of the absorbent body 10 of FIG. 1 taken along line 2B-2B.

The absorbent body 10 can have a longitudinal direction (X), a transverse direction (Y), and a depth direction (Z). The absorbent body 10 can have a first transverse direction end edge 12, a second transverse direction end edge 14 opposite the first transverse direction end edge 12, and a pair of opposing longitudinal direction side edges 16 connecting the first and second transverse direction end edges, 12 and 14. In various embodiments, the absorbent body 10 can have a first absorbent layer 20 and a second absorbent layer 22. In various embodiments, the absorbent body 10 can have a first absorbent layer 20, a second absorbent layer 22, and a third absorbent layer 24. The absorbent body 10 can have a body facing surface 26 which can be the uppermost surface of the absorbent body 10. The absorbent body 10 can have a garment facing surface 28 which can be the bottommost surface of the absorbent body 10.

In various embodiments in which the absorbent body 10 has two absorbent layers, 20 and 22, the absorbent layers, 20 and 22, can be formed of the same material. In various embodiments in which the absorbent body 10 has two absorbent layers, 20 and 22, the absorbent layers, 20 and 22, can be formed from different material. In various embodiments in which the absorbent body 10 has three absorbent layers, 20, 22, and 24, the three absorbent layers, 20, 22, and 24, can be formed of the same material. In various embodiments in which the absorbent body 10 has three absorbent layers, 20, 22, and 24, two of the absorbent layers, 20, 22, and 24, can be formed of the same material and one of the layers, 20, 22, and 24, can be formed of a different material. In various embodiments in which the absorbent body 10 has three absorbent layers, 20, 22, and 24, each of the absorbent layers, 20, 22, and 24, is formed from a material that is different from each of the other absorbent layers, 20, 22, and 24.

Each absorbent layer, 20, 22, and/or 24, present in the absorbent body 10 can generally be any single layer structure or combination of layer components, which can demonstrate some level of compressibility, conformability, be non-irritating to a wearer's skin, and capable of absorbing and retaining liquids and other body exudates. Additionally, each absorbent layer, 20, 22, and/or 24, present in the absorbent body 10 can provide additional capacity to absorb and retain body exudates such as menses.

In various embodiments, each absorbent layer, 20, 22, and/or 24, present in the absorbent body 10 can be formed from absorbent material which can include absorbent web material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In various embodiments, the absorbent web material can include a matrix of cellulosic fluff. In various embodiment, the absorbent web material can include a matrix of cellulosic fluff and can also include superabsorbent material. The cellulosic fluff can comprise a blend of wood pulp fluff. An example of a wood pulp fluff can be identified with the trade designation NB 416, available from Weyerhaeuser Corp., and is a bleached, highly absorbent wood pulp containing primarily soft wood fibers.

In various embodiments, if desired, any of the absorbent layers, 20, 22, and/or 24, present in the absorbent body 10 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and α-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent body 10 in any amount as desired.

Regardless of the combination of absorbent materials used in the absorbent layers, 20, 22, and/or 24, of the absorbent body 10, the absorbent materials can be formed into a web structure by employing various conventional methods and techniques. For example, the absorbent web structure can be formed by techniques such as, but not limited to, a dry-forming technique, an air forming technique, a wet forming technique, a foam forming technique, or the like, as well as combinations thereof. A coform nonwoven material can also be employed. Methods and apparatus for carrying out such techniques are well known in the art. Various woven fabrics and nonwoven webs can be used to construct an absorbent layer, 20, 22, and/or 24, of the absorbent body 10. For example, an absorbent layer, 20, 22, and/or 24, of the absorbent body 10 can comprise a nonwoven fabric layer composed of a meltblown or spunbond web of polyolefin or polyester filaments. Such nonwoven fabric layers may include conjugate, biconstituent and homopolymer fibers of staple or other lengths and mixtures of such fibers with other types of fibers. An absorbent layer, 20, 22, and/or 24, of the absorbent body 10 can also be a bonded card web or an airlaid web composed of natural and/or synthetic fibers. The bonded carded web may, for example, be a powder bonded carded web, an infrared bonded carded web, or a through air bonded carded web. The bonded carded webs can optionally include a mixture or blend of different fibers. The bonded carded web can have a basis weight of less than about 100 gsm, and in some embodiments, from about 10 gsm to about 40 gsm.

The shape of the absorbent body 10 can vary as desired and can comprise any one of various shapes including, but not limited to, triangular, rectangular, dog-bone and elliptical shapes. In various embodiments, the absorbent body 10 can have a shape that generally corresponds with the overall shape of the absorbent article 100 within which the absorbent body 10 is used. The dimensions of the absorbent body 10 can be substantially similar to those of the absorbent article 100, however, it will be appreciated that the dimensions of the absorbent body 10 while similar, will often be less than those of the overall absorbent article 100, in order to be adequately contained therein.

By way of example, suitable materials and/or structures for each of the absorbent layers, 20, 22, and/or 24, of the absorbent body 10 can include, but are not limited to, those described in U.S. Pat. No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al., each of which is hereby incorporated by reference thereto in its entirety.

As described above, in various embodiments, an absorbent layer, 20, 22, and/or 24, of the absorbent body 10 can be a single layer structure and can include, for example, a matrix of cellulosic fluff and superabsorbent material. In various embodiments, an absorbent body 10 can have at least two absorbent layers, such as, for example, absorbent layers 20 and 22, which can be, for example, a body facing layer and a garment facing layer. In various embodiments, the two absorbent layers, 20 and 22, can be identical to each other. In various embodiments, the two absorbent layers, 20 and 22, layers can be different from each other. In such embodiments, the two absorbent layers, 20 and 22, can provide the absorbent body 10 with different absorption properties as deemed suitable. In various embodiments, the body facing layer of the absorbent body 10 may be constructed of through-air bonded carded web material and the garment facing layer of the absorbent body 10 may be constructed of a superabsorbent polymer-containing compressed sheet. In such embodiments, the through-air bonded carded web material can have a basis weight from about 40 to about 200 gsm and the superabsorbent polymer-containing compressed sheet can be a cellulosic fluff based material that can be a combination of cellulosic pulp and SAP enclosed with a tissue carrier and having a basis weight from about 40 to about 400 gsm. In various embodiments, the body facing layer of the absorbent body 10 may be constructed of a through-air bonded carded web material and the garment facing layer of the absorbent body 10 may be constructed of a matrix of cellulosic fluff sheet. In various embodiments, the body facing layer of the absorbent body 10 may be constructed of a through-air bonded carded web material and the garment facing layer of the absorbent body 10 may be constructed of an airlaid material.

As described above, in various embodiments, an absorbent body 10 can have at least three absorbent layers, 20, 22, and 24. In various embodiments in which the absorbent body 10 has three absorbent layers, 20, 22, and 24, the three absorbent layers, 20, 22, and 24, can be formed of the same material. In various embodiments in which the absorbent body 10 has three absorbent layers, 20, 22, and 24, two of the absorbent layers, 20, 22, or 24, can be formed of the same material and one of the layers, 20, 22, or 24, can be formed of a different material. In various embodiments in which the absorbent body 10 has three absorbent layers, 20, 22, and 24, each of the absorbent layers, 20, 22, and 24, is formed from a material that is different from each of the other absorbent layers, 20, 22, and 24. In various embodiments, the first absorbent layer 20 can be constructed of a through-air bonded carded web; the second absorbent layer 22 can be constructed of a cellulosic fluff based material, a superabsorbent-polymer containing compressed sheet, a wetlaid material, such as, for example, a tissue layer, or an airlaid, material; and the third absorbent layer 24 can be constructed of a nonwoven material, a cellulosic fluff based material, a superabsorbent-polymer containing compressed sheet, an airlaid material, or a wetlaid material, such as, for example, a tissue layer. In various embodiments in which the absorbent body 10 has three absorbent layers, 20, 22, and 24, the first absorbent layer 20 can be a through-air bonded carded web, the second absorbent layer 22 can be a cellulosic fluff based material, and the third absorbent layer 24 can be a tissue layer.

In various embodiments, the absorbent body 10 can have a plurality of pairs of compressed points 30. Each pair of compressed points 30 can have a first compressed point 32 and a second compressed point 34. In various embodiments in which the absorbent body 10 can have a first absorbent layer 20 and a second absorbent layer 22, each pair of compressed points 30 can compress the first absorbent layer 20 into the second absorbent layer 22. In various embodiments, in which the absorbent body 10 has a first absorbent layer 20, a second absorbent layer 22, and a third absorbent layer 24, each pair of compressed points 30 can compress the first absorbent layer 20 into the second absorbent layer 22 and into the third absorbent layer 24. In various embodiments each pair of compressed points 30, therefore, extends into each absorbent layer, 20, 22, and/or 24 present within the absorbent body 10. In various embodiments, each compressed point, 32 and 34, extends from the body facing surface 26 of the absorbent body 10 to the garment facing surface 28 of the absorbent body 10. The presence of each compressed point, 32 and 34, within each absorbent layer, 20, 22, and/or 24, of the absorbent body 10 can provide the absorbent body 10 with vertical wicking capability as the body exudates can travel into each compressed point, 32 and 34, and penetrate deeper into the absorbent body 10. Each of the compressed points can provide a pathway for body exudates to travel from the upper surface of the absorbent body and into the lower layer(s) of the absorbent body. Providing pathways for the body exudates to travel can increase the vertical wicking capability of the absorbent body and decrease the feeling of wetness to the wearer of the absorbent article.

In various embodiments, providing an absorbent body 10 with compressed points, 32 and 34, can increased vertical wicking capability. However, if too many compressed points, 32 and 34, are incorporated into the absorbent body 10 performance of the absorbent body 10 to absorb and retain body exudates can decrease as a higher area of compressed points, 32 and 34, can result in an overall increase in the density of the absorbent body 10 and an overall decrease in the void volume of the absorbent body 10. In various embodiments, to maintain a larger void volume of the absorbent body 10 it may be desirable to have a lower area of compressed points, 32 and 34, with respect to the area of the absorbent body 10 overall. In various embodiments, the proportion of the area of compressed points, 32 and 34, with respect to the area of the absorbent body 10 can be less than about 12%. In various embodiments, the proportion of the area of compressed points, 32 and 34, with respect to the area of the absorbent body 10 can be from about 5 or 7% to about 10 or 12%. The lower the area of compressed points, 32 and 34, with respect to the area of the absorbent body 10 can also provide the absorbent body 10 with flexibility allowing the absorbent body 10, and the overall absorbent article 100, the ability to better conform to the body of the wearer during usage of the absorbent article 100.

Each compressed point, 32 and 34, can be incorporated into the absorbent body 10 by utilizing embossing pins wherein each embossing pin has the shape desired for each compressed point, 32 and 34. For example, an embossing pin can have a round shape which can produce a compressed point, 32 or 34, having a circular shape. Each compressed point, 32 and 34, can have any shape as desired such as, for example, circle, oval, square, triangle, diamond, rectangle. Each compressed point, 32 and 34, can have a size dimension small enough to provide the overall desired level of flexibility and vertical wicking capability to the absorbent body 10. In various embodiments, each compressed point, 32 and 34, can have a length in the longitudinal direction (X) of about 1 mm. In various embodiments, each compressed point, 32 and 34, can have a width in the transverse direction (Y) of about 1 mm. In various embodiments, each compressed point, 32 and 34, can have a length in the longitudinal direction (X) of about 1 mm and a width in the transverse direction (Y) of about 1 mm.

In various embodiments, the absorbent body 10 can have multiple pairs of compressed points 30. In various embodiments, a pair of compressed points 30 can be oriented in the transverse direction (Y). In such embodiments, each of the compressed points, 32 and 34, of the pair of compressed points 30 are side-by-side in the transverse direction (Y) of the absorbent body 10. In various embodiments, a pair of compressed points 30 can be oriented in the longitudinal direction (X) of the absorbent body 10. In such embodiments, each of the compressed points, 32 and 34, of the pair of compressed points 30 are side-by-side in the longitudinal direction (X) of the absorbent body 10.

In various embodiments in which the compressed points, 32 and 34, of the pair of compressed points 30 are oriented in the longitudinal direction (X), the pair of compressed points 30 can have a leading edge 36 and a trailing edge 38. The leading edge 36 of the pair of compressed points 30 can be the edge of a compressed point, such as compressed point 32, which is closest to the first transverse direction end edge 12 of the absorbent body 10. The trailing edge 38 of the pair of compressed points 30 can be the edge of the compressed point, such as compressed point 34, which is closest to the second transverse direction end edge 14 of the absorbent body 10. In various embodiments, the leading edge 36 and the trailing edge 38 of the pair of compressed points 30 can be separated by a distance, D1, which can be about 3 mm. As each of the compressed points, 32 and 34, can have a length in the longitudinal direction (X) of about 1 mm, a distance, D2, separating each of the compressed points, 32 and 34, of the pair of compressed points 30 can be about 1 mm.

In various embodiments, a first pair of compressed points 30 can be oriented in the longitudinal direction (X) and a second pair of compressed points 30 can be oriented in the longitudinal direction (X). In such embodiments, the first pair of compressed points 30 can be separated in the longitudinal direction (X) from the second pair of compressed points 30. The distance of separation, D3, in the longitudinal direction (X) between the two pairs of compressed points 30 can be measured from the trailing edge 38 of the first pair of compressed points to the leading edge 36 of the second pair of compressed points 30. The distance of separation, D3, in the longitudinal direction (X) between the two pairs of compressed points 30 oriented in the longitudinal direction can be about 3 mm. In various embodiments, the distance of separation, D3, in the longitudinal direction (X) between the two pairs of compressed points 30 oriented in the longitudinal direction (X) can be the same as the distance, D1, in the longitudinal direction (X) separating the leading edge 36 and the trailing edge 38 of a pair of compressed points 30. In various embodiments, the distance of separation, D3, in the longitudinal direction (X) between the two pairs of compressed points 30 oriented in the longitudinal direction (X) can be greater than the distance, D1, in the longitudinal direction (X) separating the leading edge 36 and the trailing edge 38 of a pair of compressed points 30. In various embodiments, the distance of separation D3 in the longitudinal direction (X) between the two pairs of compressed points 30 oriented in the longitudinal direction (X) can be 1.5 or 2 times greater than the distance D1 in the longitudinal direction (X) separating the leading edge 36 and the trailing edge 38 of a pair of compressed point 30.

In various embodiments, a first pair of compressed points 30 can be separated in the transverse direction (Y) from a second pair of compressed points 30. In such embodiments, each of the pairs of compressed points 30 can be oriented in the longitudinal direction (X). In such embodiments, the first pair of compressed points 30 can be separated from the second pair of compressed points 30 a distance, D4, in the transverse direction (Y). In various embodiments, the distance D4, can be greater than 3 mm. In various embodiments, the distance D4, can be about 3.5 mm. In various embodiments, the distance, D4, separating two pairs of compressed points 30 in the transverse direction (Y) can be greater than the distance, D3, separating two pairs of compressed points 30 in the longitudinal direction (X) of the absorbent body 10. In various embodiments, the distance D4, separating two pairs of compressed points 30 in the transverse direction (Y) can be the same as the distance D3 separating two pairs of compressed points 30 in the longitudinal direction (X) of the absorbent body 10.

In various embodiments, a first pair of compressed points 30 can be oriented in the longitudinal direction (X) and a second pair of compressed points 30 can be oriented in the longitudinal direction (X) and be separated from the first pair of compressed points 30 in the transverse direction (Y) by a distance, D4. In various embodiments, the second pair of compressed points 30 can be aligned with the first pair of compressed points 30 such that an invisible line drawn through both pairs of compressed points 30 in the transverse direction (Y) will intersect each pair of compressed points 30 at the same location within each pair of compressed points 30. In various embodiments, the second pair of compressed points 30 can be offset from the first pair of compressed points 30 such that an invisible line drawn through both pairs of compressed points 30 in the transverse direction (Y) will intersect each pair of compressed points 30 at different locations within each pair of compressed points 30. In various embodiments, a pattern of pairs of compressed points 30 in which one pair of compressed points 30 is offset from another pair of compressed points 30 in the transverse direction (Y) can more effectively contain the body exudates in a localized area of the absorbent body 10 thereby reducing spread of the body exudates in at least the transverse direction of the absorbent article which can result in a reduction of incidents of leakage of body exudates from the absorbent article.

### Absorbent Article:

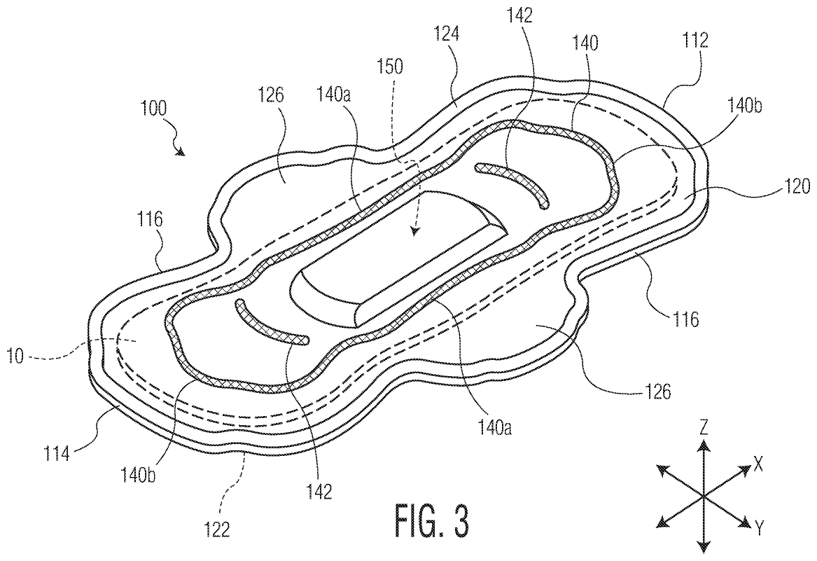

The absorbent body 10 of the present disclosure can be incorporated into an absorbent article 100. Referring to FIG. 3, FIG. 3 provides an illustration of a perspective view of an exemplary absorbent article 100. The absorbent article 100 can have a longitudinal direction (L), a transverse direction (T), and a depth direction (Z). The absorbent article 100 can have a first transverse direction end edge 112, a second transverse direction end edge 114 opposite the first transverse direction end edge 112, and a pair of opposing longitudinal direction side edges 116. In various embodiments, the absorbent article 100 can take on various geometries but will generally have a pair of opposing longitudinal direction side edges 116 and a pair of opposing transverse direction end edges 112 and 114. The absorbent article 10 can have a wearer facing, liquid permeable topsheet layer 120 and a garment facing, liquid impermeable backsheet layer 122. An absorbent body 10 can be positioned between the topsheet layer 120 and the backsheet layer 122.

The topsheet layer 120 and the backsheet layer 122 can both extend beyond the outermost peripheral edges of the absorbent body 10 and can be peripherally bonded together, either entirely or partially, using known bonding techniques to form a sealed peripheral region 124. For example, the topsheet layer 120 and the backsheet layer 122 can be bonded together by adhesive bonding, ultrasonic bonding, or any other suitable bonding method known in the art.

In various embodiments, the absorbent article 100 can have a pair of wings 126 extending outwardly, in the transverse direction T, from the absorbent article 100. The wings 126 can drape over the edges of the wearer's undergarment so that the wings 126 are disposed between the edges of the wearer's undergarment and her thighs. The wings 126 can serve at least two purposes. First, the wings 126 can prevent soiling of the wearer's undergarment by forming a barrier along the edges of the undergarment. Second, the wings 126 can be provided with an attachment aid (not shown), such as, for example, a garment attachment adhesive or a hook, to keep the absorbent article 100 securely and properly positioned in the undergarment. The wings 126 can wrap around the crotch region of the wearer's undergarment to aid in securing the absorbent article 100 to the wearer's undergarment when in use. Each wing 126 can fold under the crotch region of the wearer's undergarment and the attachment aid can either form a secure attachment to the opposite wing 126 or directly to the surface of the wearer's undergarment. In various embodiments, the wings 126 can be an extension of materials forming the topsheet layer 120 and/or the backsheet layer 122, such that the wings 126 can be of a unitary construction with the absorbent article 100. In various embodiments, the wings 126 can be constructed of materials similar to the topsheet layer 120, the backsheet layer 122 or combinations of these materials. In various embodiments, the wings 126 can be separate elements bonded to the main body of the absorbent article 100. It is to be understood that the wings 126 are optional and, in various embodiments, an absorbent article 100 can be configured without wings 126.

### Topsheet Layer:

The topsheet layer 120 defines a wearer facing surface of the absorbent article 100 that may directly contact the body of the wearer and is liquid permeable to receive body exudates. The topsheet layer 120 is desirably provided for comfort and conformability and functions to direct body exudates away from the body of the wearer, through its own structure, and towards the absorbent body 10. The topsheet layer 120 desirably retains little to no liquid in its structure, so that it provides a relatively comfortable and non-irritating surface next to the skin of the wearer of the absorbent article 100.

The topsheet layer 120 can be a single layer of material, or alternatively, can be multiple layers that have been laminated together. The topsheet layer 120 can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a topsheet layer 120.

In various embodiments, the topsheet layer 120 can be constructed from various nonwoven webs such as meltblown webs, spunbond webs, hydroentangled spunlace webs, or through air bonded carded webs. Examples of suitable topsheet layer 120 materials can include, but are not limited to, natural fiber webs (such as cotton), rayon, hydroentangled webs, bonded carded webs of polyester, polypropylene, polyethylene, nylon, or other heat-bondable fibers (such as bicomponent fibers), polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable topsheet layer 120 can be a bonded carded web made of polypropylene and polyethylene such as that obtainable from Sandler Corporation, Germany. U.S. Pat. No. 4,801,494 to Datta, et al., and U.S. Pat. No. 4,908,026 to Sukiennik, et al., and WO 2009/062998 to Texol teach various other topsheet materials that may be used as the topsheet layer 120, each of which is hereby incorporated by reference thereto in its entirety. Additional topsheet layer 120 materials can include, but are not limited to, those described in U.S. Pat. No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 120 may contain a plurality of apertures (not shown) formed therethrough to permit body exudates to pass more readily into the absorbent body 10. The apertures may be randomly or uniformly arranged throughout the topsheet layer 120. The size, shape, diameter, and number of apertures may be varied to suit an absorbent article's 100 particular needs.

In various embodiments, the topsheet layer 120 can have a basis weight ranging from about 5, 10, 15, 20 or 25 gsm to about 50, 100, 120, 125 or 150 gsm. For example, in an embodiment, a topsheet layer 120 can be constructed from a through air bonded carded web having a basis weight ranging from about 15 gsm to about 100 gsm. In another example, a topsheet layer 120 can be constructed from a through air bonded carded web having a basis weight from about 20 gsm to about 50 gsm, such as a through air bonded carded web that is readily available from nonwoven material manufacturers, such as Xiamen Yanjan Industry, Beijing, DaYuan Nonwoven Fabrics and others.

In various embodiments, the topsheet layer 120 can be at least partially hydrophilic. In various embodiments, a portion of the topsheet layer 120 can be hydrophilic and a portion of the topsheet layer 120 can be hydrophobic. In various embodiments, the portions of the topsheet layer 120 which can be hydrophobic can be either an inherently hydrophobic material or can be a material treated with a hydrophobic coating.

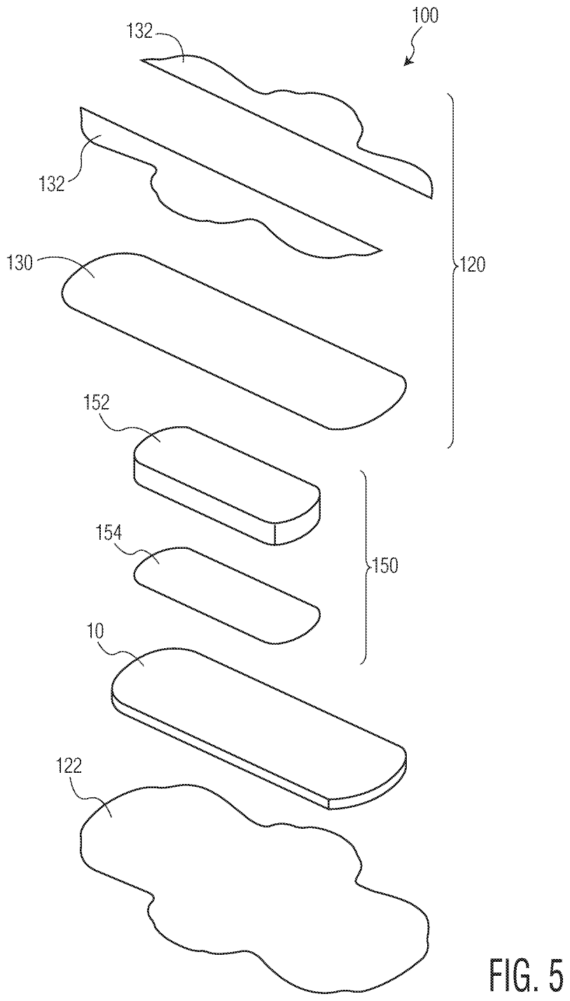

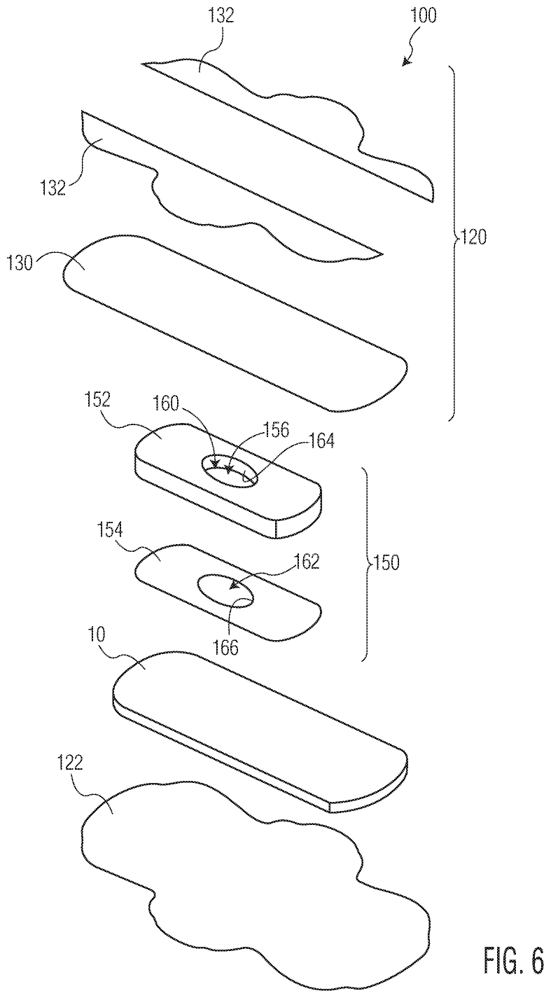

In various embodiments, the topsheet layer 120 can be a multicomponent topsheet layer 120 such as by having two or more different nonwoven or film materials, with the different materials placed in separate locations in the transverse direction T of the absorbent article 100. For example, referring to FIGS. 5 and 6, the topsheet layer 120 can be a two layer or multicomponent material having a central portion 130 positioned along and straddling a longitudinal centerline of the absorbent article 100, with lateral side portions 132 flanking and bonded to each side edge of the central portion 130. The central portion 130 can be constructed from a first material and the side portions 132 can be constructed from a material which can be the same as or different from the material of the central portion 130. In such embodiments, the central portion 130 may be at least partially hydrophilic and the side portions may be inherently hydrophobic or may be treated with a hydrophobic coating. Examples of constructions of multi-component topsheet layers 120 are generally described in U.S. Pat. No. 5,961,505 to Coe, U.S. Pat. No. 5,415,640 to Kirby, and U.S. Pat. No. 6,117,523 to Sugahara, each of which is incorporated herein by reference thereto in its entirety.

In various embodiments, a central portion 130 of a topsheet layer 120 can be positioned symmetrically about the absorbent article 100 longitudinal centerline. Such central longitudinally directed central portion 130 can be a through air bonded carded web (“TABCW”) having a basis weight between about 15 and about 100 gsm. Previously described nonwoven, woven, and apertured film topsheet layer materials may also be used as the central portion 130 of a topsheet layer 120. In various embodiments, the central portion 130 can be constructed from a TABCW material having a basis weight from about 20 to about 50 gsm such as is available from Xiamen Yanjan Industry, Beijing, DaYuan Nonwoven Fabrics, and others. Alternatively, apertured films, such as those available from such film suppliers as Texol, Italy and Tredegar, U.S.A. may be utilized. Different nonwoven, woven, or film sheet materials may be utilized as the side portions 132 of the topsheet layer 120. The selection of such topsheet layer 120 materials can vary based upon the overall desired attributes of the topsheet layer 120. For example, it may be desired to have a hydrophilic material in the central portion 130 and hydrophobic-barrier type materials in the side portions 132 to prevent leakage and increase a sense of dryness in the area of the side portions 132. Such side portions 132 can be adhesively, thermally, ultrasonically, or otherwise bonded to the central portion 130 along or adjacent the longitudinally directed side edges of the central portion 130. Traditional absorbent article construction adhesive may be used to bond the side portions 132 to the central portion 130. Either of the central portion 130 and/or the side portions 132 may be treated with surfactants and/or skin-health benefit agents, as are well known in the art.

Such longitudinally directed side portions 132 can be of a single or multi-layered construction. In various embodiments, the side portions 132 can be adhesively or otherwise bonded laminates. In various embodiments, the side portions 132 can be constructed of an upper fibrous nonwoven layer, such as a spunbond material, laminated to a bottom layer of a hydrophobic barrier film material. Such a spunbond layer may be formed from a polyolefin, such as a polypropylene and can include a wetting agent if desired. In various embodiments, a spunbond layer can have a basis weight from about 10 or 12 gsm to about 30 or 70 gsm and can be treated with hydrophilic wetting agents. In various embodiments, a film layer may have apertures to allow fluid to permeate to lower layers, and may be either of a single layer or multi-layer construction. In various embodiments, such film can be a polyolefin, such as a polyethylene having a basis weight from about 10 to about 40 gsm. Construction adhesive can be utilized to laminate the spunbond layer to the film layer at an add-on level of between about 0.1 gsm and 15 gsm. When a film barrier layer is used in the overall topsheet layer 120 design, it may include opacifying agents, such as film pigments, that can help the film in masking stains along the absorbent article 10 side edges, thereby serving as a masking element. In such a fashion, the film layer can serve to limit visualization of a fluid insult stain along the absorbent article 100 side edges when viewed from above the topsheet layer 120. The film layer may also serve as a barrier layer to prevent rewet of the topsheet layer 120 as well as to prevent the flow of fluid off the side edges of the absorbent article 100. In various embodiments, the side portions 132 can be laminates such as a spunbond-meltblown-meltblown-spunbond layer (“SMMS”) laminate, spunbond-film laminate, or alternatively, other nonwoven laminate combinations.

### Backsheet Layer:

The backsheet layer 122 is generally liquid impermeable and is the portion of the absorbent article 100 which faces the garment of the wearer. The backsheet layer 122 can permit the passage of air or vapor out of the absorbent article 10 while still blocking the passage of liquids. Any liquid impermeable material may generally be utilized to form the backsheet layer 122. The backsheet layer 122 can be composed of a single layer or multiple layers, and these one or more layers can themselves comprise similar or different materials. Suitable material that may be utilized can be a microporous polymeric film, such as a polyolefin film of polyethylene or polypropylene, nonwovens and nonwoven laminates, and film/nonwoven laminates. The particular structure and composition of the backsheet layer 122 can be selected from various known films and/or fabrics with the particular material being selected as appropriate to provide the desired level of liquid barrier, strength, abrasion resistance, tactile properties, aesthetics and so forth. In various embodiments, a polyethylene film can be utilized that can have a thickness in the range of from about 0.2 or 0.5 mils to about 3.0 or 5.0 mils. An example of a backsheet layer 122 can be a polyethylene film such as that obtainable from Pliant Corporation, Schaumburg, Ill., USA. Another example can include calcium carbonate-filled polypropylene film. In still another embodiment, the backsheet layer 122 can be a hydrophobic nonwoven material with water barrier properties such as a nonwoven laminate, an example of which can be a spunbond, meltblown, meltblown, spunbond, four-layered laminate. The backsheet layer 122 can, therefore, be of a single or multiple layer construction, such as of multiple film layers or laminates of film and nonwoven fibrous layers. Suitable backsheet layers 122 can be constructed from materials such as those described in U.S. Pat. No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

### Fluid Intake Layer:

In various embodiments, the absorbent article 100 can include a liquid permeable fluid intake layer 150 positioned between the topsheet layer 120 and the absorbent body 10. Such a fluid intake layer 150 can be made of a material that can be capable of rapidly transferring, in the depth direction (Z), body exudates that are delivered to the topsheet layer 120. The fluid intake layer 150 can generally have any shape and/or size desired. In an embodiment, the fluid intake layer 150 can have a curved rectangular shape, with a length equal to or less than the overall length of the absorbent article 100, and a width less than the width of the absorbent article 100. For example, the fluid intake layer 150 can have a length of between about 20, 40 or 60 mm to about 150, 150, 175, 200 or 300 mm and a width of between about 10, 15 or 20 mm to about 60, 80 or 100 mm may be utilized. In various embodiments, the fluid intake layer 150 can have a height, in the depth direction (Z), from about 1, 2, 3, 4, or 5 mm to about 5.5, 6, 8, 10, 13, or 15 mm.

Any of a variety of different materials can be capable of being used for the fluid intake layer 150 to accomplish the above-mentioned functions. The material may be synthetic, cellulosic, or a combination of synthetic and cellulosic materials. The fluid intake layer 150 can be constructed from any woven or nonwoven material. For example, the fluid intake layer 150 can be constructed as an airlaid or TABCW material. For example, airlaid cellulosic tissues may be suitable for use in the fluid intake layer 150. The airlaid cellulosic tissue may have a basis weight ranging from about 10 or 100 gsm to about 250 or 300 gsm. The airlaid cellulosic tissue can be formed from hardwood and/or softwood fibers. An airlaid cellulosic tissue can have a fine pore structure and can provide an excellent wicking capacity, especially for menses.

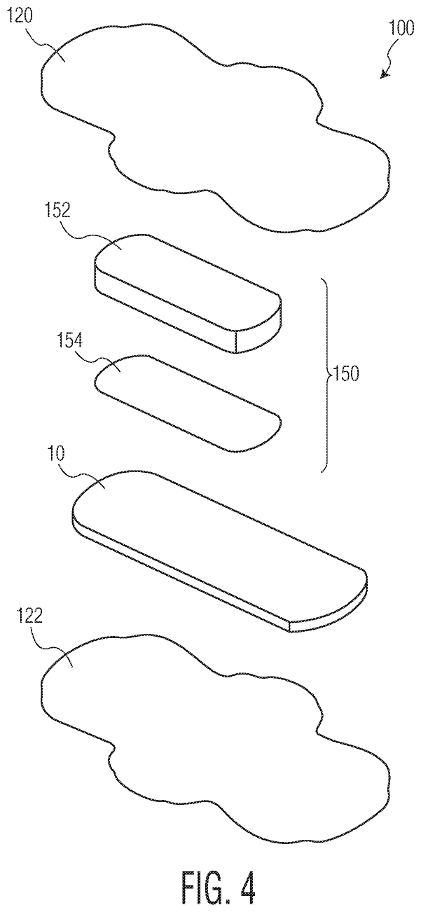

In various embodiments, the fluid intake layer 150 can be constructed as a multi-layer component of the absorbent article 100 formed from multiple layers of nonwoven material which are bonded together. For example, such as illustrated in FIGS. 4-6, the fluid intake layer 150 can have two material layers such as a first material layer 152 and a second material layer 154. The first material layer 152 can be positioned between the topsheet layer 120 and the second material layer 154 in the depth direction (Z) of the fluid intake layer 150 and the second material layer 154 can be positioned between the first material layer 152 and the absorbent body 10 in the depth direction (Z) of the fluid intake layer 150. In various embodiments, the fluid intake layer 150 can be formed of two material layers of nonwoven material bonded together. In various embodiments, the fluid intake layer 150 can be formed of more than two material layers of nonwoven material bonded together. In various embodiments, the fluid intake layer 150 can be formed of at least 2, 3, 4, or 5 material layers of nonwoven material. In various embodiments, the material forming each of the first material layer 152 and the second material layer 154 can be different from each other. For example, the fluid intake layer 150 can have a first material layer 152 constructed of a through-air bonded carded web material and a second material layer 154 constructed of an airlaid cellulosic material. In such an example, the two material layers, 152 and 154, can be bonded together to form the fluid intake layer 150. In such an example, the airlaid cellulosic material 154 can have a basis weight ranging from about 10 or 100 gsm to about 250 or 300 gsm. The airlaid cellulosic material can be formed from hardwood and/or softwood fibers. The airlaid cellulosic tissue can have a fine pore structure and can provide an excellent wicking capacity, especially for menses. In such an example, the through-air bonded carded web can have a basis weight ranging from about 10 or 100 gsm to about 150, 200, 250, or 300 gsm.

Additionally, to further enhance the ability of the absorbent article 10 to transfer body exudates in the depth (Z) direction from the topsheet layer 120 toward any lower layers in the absorbent article 100 as well as to enhance the ability of the fluid intake layer 150 to conform to the wearer's body based on its ability to bend, the fluid intake layer 150 can have a fluid intake layer opening 156 (such as illustrated, for example, in FIG. 6) which can be any suitable shape, such as ovular, circular, rectangular, square, triangular, etc. In various embodiments, the fluid intake layer opening 156 can be elongate and can be oriented in the longitudinal direction of the absorbent article 100. To form the opening 156 in the fluid intake layer 150, each of the first material layer 152 and the second material layer 154 of the fluid intake layer 150 can have an opening, 160 and 162, respectively. The opening 160 of the first material layer 152 can be bounded by a perimeter 164 which can form an inner border or inner edge of the first material layer 152 of the fluid intake layer 150. The opening 162 of the second material layer 154 can be bounded by a perimeter 166 which can form an inner border of inner edge of the second material layer 154 of the fluid intake layer 150. The opening 156 of the fluid intake layer 150 can leave a portion of the absorbent body 10 exposed and visible due to the lack of presence of the material, i.e., a void space, of the fluid intake layer 150 at the fluid intake layer opening 156.

The fluid intake layer opening 156 can be located at various positions along the longitudinal and transverse directions of the fluid intake layer 150 depending upon the primary location of body exudate intake or the purpose for which the absorbent article 100 is being used. For example, in various embodiments, the fluid intake layer 150 and the fluid intake layer opening 156 can be positioned so that it is in substantial alignment with the longitudinal centerline and the transverse centerline of the absorbent article 100. This allows the fluid intake layer opening 156 to be centrally disposed so that it can be positioned below the main point of body exudate discharge and so that it can act as the primary body exudate receiving area for the absorbent article 100.

However, centralized positioning of the fluid intake layer 150 and the fluid intake layer opening 156 is not required, and in various embodiments, depending on the primary location where body exudate intake might occur, the fluid intake layer 150 and the fluid intake layer opening 156 may be substantially aligned with the longitudinal centerline only. Thus, in various embodiments, the fluid intake layer 150 and the fluid intake layer opening 156 may be shifted in the longitudinal direction towards either transverse direction end edge, 112 or 114, of the absorbent article 100, so that the fluid intake layer opening 156 is not in substantial alignment with the transverse centerline of the absorbent article 100.

The fluid intake layer opening 156 can have a longitudinal length from about 15, 20, 30 or 50 mm to about 60, 75, 100 or 150 mm and can have a transverse width from about 10, 15, 20 or 30 mm to about 40, 60 or 80 mm. The fluid intake layer opening 156 can have a longitudinal length that is from about 15, 20 or 25% to about 70, 75, or 80% of the overall longitudinal length of the fluid intake layer 150 in the longitudinal direction. The fluid intake layer opening 156 can have a transverse width that can be from about 20, 25 or 30% to about 70, 75 or 80% of the overall width of the fluid intake layer 150 in the transverse direction. The fluid intake layer opening 156 can serve to funnel and direct body exudates from the topsheet layer 120 and towards the absorbent body 10 in the depth (Z) direction. The fluid intake layer opening 156 can also form a cup or well-like structure for holding body exudates and preventing its leakage away from a central region of the absorbent article 100 and towards the edges of the absorbent article 100.

### Embossing:

In various embodiments, the absorbent article 100 can have one or more embossed regions formed in one or more layers of the absorbent article 100. Generally, the embossed regions can be described as channels formed in the absorbent article 100 due to deformations of the layer(s) of the absorbent article 100. The embossed regions are formed in any suitable pattern to not only create an aesthetically pleasing surface, but also to facilitate funneling of body exudates towards a desired location in the absorbent article 100. The embossed regions may also improve the consistency of the fit properties of the absorbent article 100, both before and after receiving body exudates. The embossed regions may be provided in either a symmetric or asymmetric manner to the absorbent article 100.

Further, the embossed regions can be formed using any known conventional technique known in the art. Suitable techniques includes, for example, the use of raised elements to impart the desired embossing pattern to create compressed channels in the layer(s) of the absorbent article 100. For instance, a suitable process may include using thermal bonding wherein the absorbent article 100 is passed through two rolls (e.g., steel, rubber, etc.) where one is engraved with an embossing pattern and the other is flat. One or both rolls may be heated. In addition, thermal and/or ultrasonic bonding techniques may be employed to create the embossing regions.

The absorbent article 100 can have at least one embossed region 140. The embossed region 140 can provide both an aesthetically pleasing appearance and topography to the absorbent article 100 which can direct body exudates to a desired location and prevent leakage and/or pooling of the body exudate around the edge of the absorbent article 100.

In various embodiments, the embossed region 140 can generally extend around the entire absorbent article 100 in a generally oblong shape. In various embodiments, the embossed region 140 can have a protrusion(s) in the longitudinal and/or transverse directions of the absorbent article 100 to provide any additional desired shaping to the embossed region 140. FIG. 3 provides an exemplary illustration of an embossed region 140 in which the embossed region 140 has an opposing pair of longitudinally extending side portions 140a which extend the longitudinal length of the absorbent article 100. The embossed region 140 also has an opposing pair of transversely extending end portions 140b which are present in proximity to the transverse end edges, 112 and 114, of the absorbent article 100 and which connect the longitudinally extending side portions 140a. While the embossed region 140 is illustrated as extending the majority of the absorbent article 100 longitudinal length, it is to be understood that the embossed region 140 can be any size and shape as desired and can be located in only the central region of the absorbent article 100 or can extend into either or both end region of the absorbent article 100.

In various embodiments, the absorbent article 100 can have one or more second embossed regions 142. A second embossed region 142 can be positioned in either or both end region of the absorbent article 100. In the exemplary illustration of FIG. 3, the second embossed regions 142 can have a semi-circular shape, although any other suitable shape can be used.

### Wings:

The wings 126 can be constructed from materials described above with respect to the topsheet layer 120 and the backsheet layer 122. In various embodiments, the wings 126 can comprise an extension of a layer of material within the topsheet layer 120 and/or the backsheet layer 122. By way of example, the wings 126 can be formed by an extension of the topsheet layer 120 and backsheet layer 122 that are then bonded together along peripheral seal 124. Such wings 126 can be integrally formed with the main portion of the absorbent article 100. Alternatively, the wings 126 can be formed independently and separately attached to an intermediate section of the absorbent article 100. Wings 126 that are made independent of the other components of the absorbent article 100 can be bonded to a portion of the topsheet layer 120 and/or backsheet layer 122. Examples of processes for manufacturing absorbent articles 100 and wings 126 include, but are not limited to, those described in U.S. Pat. No. 4,059,114 to Richards, U.S. Pat. No. 4,862,574 to Hassim, et al., U.S. Pat. No. 5,342,647 to Heindel, et al., U.S. Pat. No. 7,070,672 to Alcantara, et al., U.S. Publication No., 2004/0040650 to Venturino, et al., and international publication WO1997/040804 to Emenaker, et al., each of which are hereby incorporated by reference thereto in its entirety.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent body comprising:
a. a longitudinal direction and a transverse direction;
b. a first absorbent layer;
c. a second absorbent layer;
d. a first pair of compressed points separated in the longitudinal direction from a second pair of compressed points by a first distance;
e. a third pair of compressed points separated in the transverse direction from the first pair of compressed points by a second distance.

2. The absorbent body of claim 1 wherein the second distance is the same as the first distance.

3. The absorbent body of claim 1 wherein the second distance is greater than the first distance.

4. The absorbent body of claim 1 wherein each compressed point in the first pair of compressed points is separated from each other by a third distance which is smaller than the first distance.

5. The absorbent body of claim 1 further comprising a third absorbent layer.

6. The absorbent body of claim 1 wherein the first pair of compressed points are oriented in the longitudinal direction.

7. The absorbent body of claim 1 wherein the second pair of compressed points are oriented in the longitudinal direction.

8. The absorbent body of claim 1 wherein the third pair of compressed points are offset from the first pair of compressed points.

9. The absorbent body of claim 1 wherein the first pair of compressed points has a leading edge and a trailing edge separated by a fourth distance.

10. The absorbent body of claim 9 wherein the fourth distance is the same as the first distance.

11. The absorbent body of claim 9 wherein the first distance is greater than the fourth distance.

12. The absorbent body of claim 1 wherein the proportion of the area of the compressed points with respect to the area of the absorbent body is less than about 12%.

13. The absorbent body of claim 5 wherein the first absorbent layer is a through-air bonded carded web, the second absorbent layer is a cellulosic fluff based material, and the third absorbent layer is a tissue layer.

14. The absorbent body of claim 1 wherein each compressed point of the first pair of compressed points, the second pair of compressed points, and the third pair of compressed points extends from an uppermost surface of the absorbent body to a bottommost surface of the absorbent body.

15. An absorbent article comprising:
a. A topsheet layer;
b. A backsheet layer; and
c. The absorbent body of claim 1.

16. The absorbent article of claim 15 further comprising a fluid intake layer positioned between the absorbent body and the topsheet layer.
